(19) United States
(12) Patent Application Publication
(10) Pub. No.: US 2018/0249936 A1
(43) Pub. Date: Sep. 6, 2018

(54) PULSE OXIMETER WITH ADDED CONTEXT ON PATIENT MONITOR

(71) Applicant: KONINKLIJKE PHILIPS N.V., EINDHOVEN (NL)

(72) Inventors: John CRONIN, BONITA SPRINGS, FL (US); Joseph BODKIN, FORT MEYERS, FL (US)

(21) Appl. No.: 15/759,247

(22) PCT Filed: Sep. 20, 2016
(86) PCT No.: PCT/EP2016/072214

§ 371 Date: Mar. 12, 2018

Related U.S. Application Data
(60) Provisional application No. 62/222,549, filed on Sep. 23, 2015.

(30) Foreign Application Priority Data
Mar. 18, 2016 (EP) 16161266.8

(51) Int. Cl. A61B 5/1455; A61B 5/0205; A61B 5/026; A61B 5/00; G16H 10/60; G16H 20/10

(52) U.S. Cl. CPC A61B 5/14552; A61B 5/02427; A61B 5/0261; A61B 5/0022; A61B 5/7435; A61B 5/743; A61B 5/7425; A61B 5/7475; A61B 5/7275; A61B 5/725; A61B 5/7445; A61B 5/7282; A61B 5/7246; G16H 10/60; G16H 20/10; A61B 5/0205

(57) ABSTRACT

Systems and methods for comparing a first patient's pulse oximeter data with a second patient's pulse oximeter data. The system of the present invention comprises a pulse oximeter device and a remote server. The method of the present invention comprises acquiring a first patient's pulse oximeter data and inputting one or more markers for various segments of the first patient's acquired pulse oximeter data. A second patient's pulse oximeter data corresponding to a known condition may be displayed with the first patient's pulse oximeter data and the one or more markers to aid the medical personnel in diagnosing the first patient's condition. After diagnosis, the first patient's pulse oximeter may also be simplified to allow the first patient to understand the pulse oximeter data.

### BACKGROUND OF THE INVENTION

Pulse oximetry is an effective and non-invasive method of acquiring oxygen saturation (SpO2) level and perfusion index of a patient. The medical personnel must then carefully assess the patient's pulse oximeter data to determine the patient's health status. In determining the patient's health status, the medical personnel may also analyze the correlation of the pulse oximeter data with other physiological parameters of the patient, such as body temperature. In addition, it may be helpful for the medical personnel if the patient's pulse oximeter data is compared with another patient's pulse oximeter data, in which the health status is already known. In this way, the medical personnel may initially diagnose the patient's condition due to the similarities between the patient's pulse oximeter data and another patient's pulse oximeter data having a known condition.

### SUMMARY OF THE CLAIMED INVENTION

Some embodiments of the present invention relate to systems and methods for comparing a first patient's pulse oximeter data with a second patient's pulse oximeter data previously stored in a database. The system comprises a pulse oximeter device and a remote server. The method comprises acquiring a first patient's pulse oximeter data and inputting one or more markers for various segments of the first patient's acquired pulse oximeter data, wherein the one or more markers correspond to a period beginning with the time of acquisition of patient context data. A second patient's pulse oximeter data corresponding to a known condition may be displayed with the first patient's pulse oximeter data and the one or more markers to aid the medical personnel in diagnosing the first patient's condition. After diagnosis, the first patient's pulse oximeter may also be simplified to allow the first patient to understand the pulse oximeter data. The database may then be updated by uploading data corresponding to the diagnosed first patient's condition, the first patient's pulse oximeter data with the one or more markers, and the simplified first patient's pulse oximeter data.

### BRIEF DESCRIPTION OF THE DRAWINGS

FIG. 1 illustrates an overall system for comparing a first patient's pulse oximeter data with a second patient's pulse oximeter data according to some embodiments.

FIG. 2 illustrates the process of comparing a first patient's pulse oximeter data with a second patient's pulse oximeter data according to some embodiments.

FIG. 3A-3E illustrates some embodiments of the graphical user interface of the pulse oximeter device.

FIG. 4A illustrates the “Context Data” display mode of the graphical user interface.

FIG. 4B illustrates the “Time Context” display mode of the graphical user interface.

FIG. 4C illustrates the “Comparison Context” display mode of the graphical user interface.

FIG. 4D illustrates the “User View” display mode of the graphical user interface.

### DETAILED DESCRIPTION OF THE EMBODIMENTS

Some embodiments of the present invention relate to a system for comparing a first patient's pulse oximeter data with a second patient's pulse oximeter data previously stored in a database, the system comprising: a database for storing data corresponding to a diagnosed first patient's condition, the first patient's acquired pulse oximeter data with the one or more markers, and the simplified first patient's acquired pulse oximeter data; and a pulse oximeter device for acquiring a first patient's pulse oximeter data comprising; a display with a user interface for inputting one or more markers for various segments of the first patient's acquired pulse oximeter data; and a processor for executing an algorithm to simplify the first patient's acquired pulse oximeter data to allow the first patient to understand the acquired pulse oximeter data.

Some embodiments of the present invention also relates to a method for comparing a first patient's pulse oximeter data with a second patient's pulse oximeter data previously stored in a database comprising: acquiring a first patient's pulse oximeter data using a pulse oximeter; inputting via a user interface one or more markers for various segments of the first patient's acquired pulse oximeter data, wherein the one or more markers correspond to a period beginning with the time of acquisition of patient context data; displaying the first patient's acquired pulse oximeter data with the one or more markers; selecting via the user interface the second patient's pulse oximeter data previously stored in the database, wherein the second patient's pulse oximeter data corresponds to a known condition; diagnosing a first patient's condition based on the first patient's acquired pulse oximeter data with the one or more markers and the second patient's pulse oximeter data; simplifying the first patient's acquired pulse oximeter data to allow the first patient to understand the acquired pulse oximeter data; displaying the diagnosed first patient's condition and the simplified first patient's acquired pulse oximeter data; and updating the database by uploading data corresponding to the diagnosed first patient's condition, the first patient's acquired pulse oximeter data with the one or more markers, and the simplified first patient's acquired pulse oximeter data.

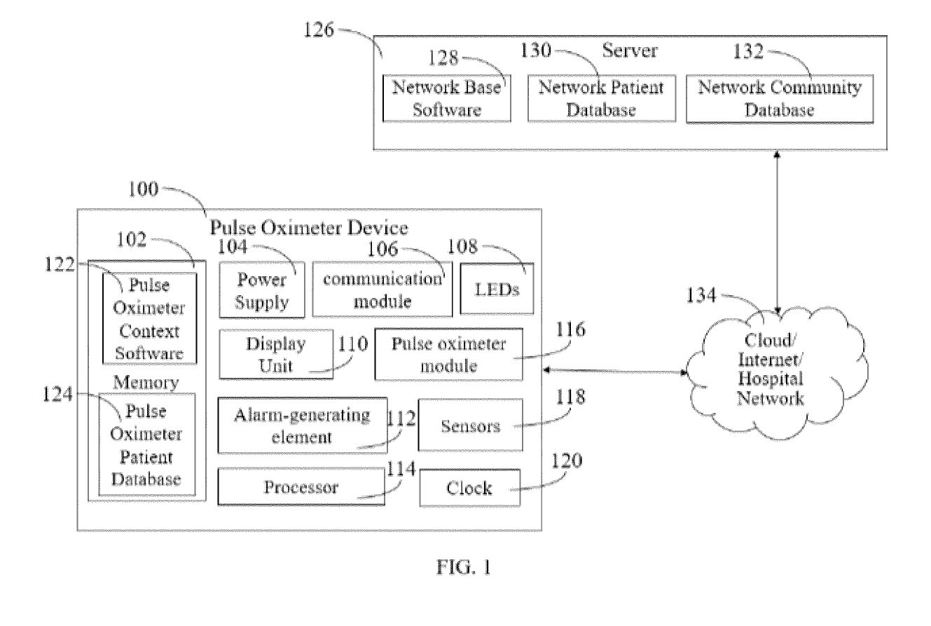

FIG. 1 illustrates a preferred embodiment of the system directed to diagnosing a first patient's condition by comparing a first patient's pulse oximeter data with a second patient's pulse oximeter data. The pulse oximeter device 100 comprises a memory 102, a power supply 104, a communication module 106, one or more light emitting diodes (LEDs) 108, a display unit 110, an alarm-generating element 112, a processor 114, a pulse oximeter module 116, one or more sensors 118, and a clock 120. The memory 102 further comprises a pulse oximeter context software 122 and a pulse oximeter patient database 124. The pulse oximeter device 100 is preferably connected to a remote server 126 that comprises a network base software 128, a network patient database 130, and a network community database 132. The cloud network 134 (e.g., the cloud, the internet, a hospital network) allows the communication between the remote server 126 and the pulse oximeter device 100.

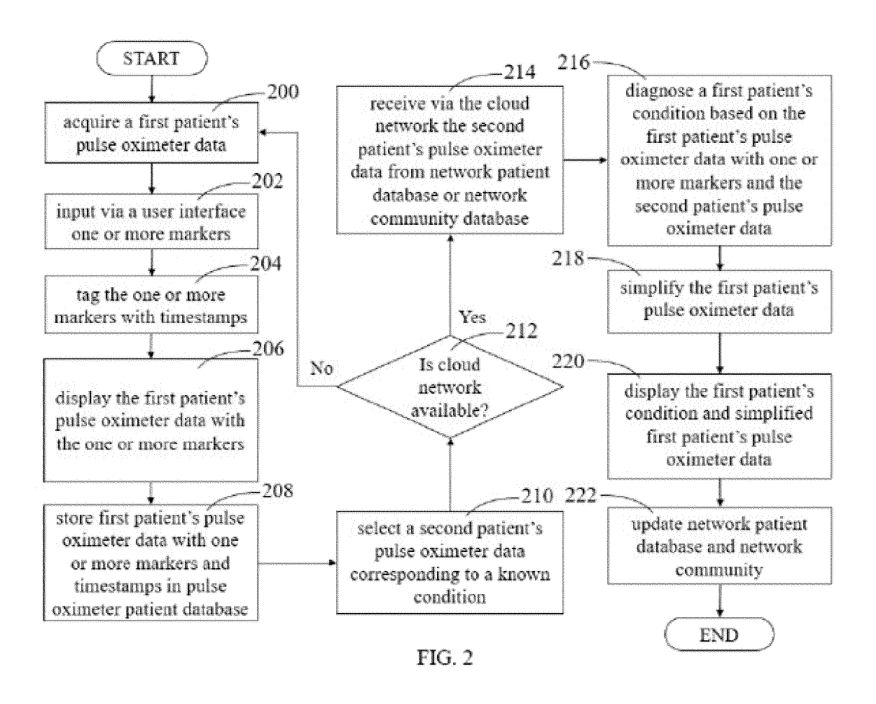

FIG. 2 illustrates a preferred embodiment of the method of the present invention. A pulse oximeter module may be attached to a patient's body part, preferably the patient's finger, to acquire pulse oximeter data, such as blood oxygen saturation levels, perfusion index, and pulse rate (step 200). The first patient's pulse oximeter data may then be displayed, preferably plotted in the form of a time-dependent graph. Upon viewing the graph of the pulse oximeter data, a user (e.g., medical personnel) may input via the graphical user interface one or more markers and their corresponding time periods (timestamps) (steps 202 and 204). The one or more markers may indicate patient's context data, which may include, but is not limited to, type and quantity of food, drug, and fluid administered to the patient. For example, while the time-dependent graph of the patient's blood oxygen saturation levels is being displayed, the user may choose to select the marker representing anti-arrhythmic medicine and the time period that the anti-arrhythmic medicine is administered to the patient. Subsequently, the time-dependent graph for the patient's blood oxygen saturation levels is displayed with the marker overlaying the time-dependent graph to allow the medical personnel to analyze the correlation and possible effects of the anti-arrhythmic medicine to the patient's blood oxygen saturation levels (step 206). In an alternate embodiment of the invention, the marker is automatically tagged with a corresponding timestamp upon selection of the marker via the graphical user interface.

After inputting the one or more markers and the corresponding timestamps via the graphical user interface, the first patient's pulse oximeter data with one or more markers and timestamps are then stored in the memory's pulse oximeter patient database (step 208). By using the graphical user interface, the user may also select a second patient's pulse oximeter data, which corresponds to a known condition (step 210). The pulse oximeter device then determines if it can communicate to the cloud network (step 212). If the cloud network is unavailable, which may be due to loss of Internet connection, the pulse oximeter device may continue to acquire pulse oximeter data from the patient. If the cloud network is available, the pulse oximeter device may receive the second patient's pulse oximeter data stored in the network patient database or network community database (step 214).

Thereafter, the second patient's pulse oximeter data may be displayed along with the first patient's pulse oximeter data with one or more markers. This helps the medical personnel in diagnosing the first patient's condition by comparing the first patient's pulse oximeter data with the second patient's pulse oximeter data having a known condition (step 216). For example, the medical personnel may provide an initial diagnosis that the first patient may be experiencing a chronic obstructive pulmonary disease based on the correlation between the first patient's pulse oximeter data and the second patient's pulse oximeter data with a determined chronic obstructive pulmonary disease. The medical personnel may also choose to analyze manually the first patient's pulse oximeter data with the first patient's medical records and other physiological data—which may include body temperature, glucose level, and sweat electrolyte level—to provide a conclusive diagnosis of the first patient. The medical personnel may then use the graphical user interface to input the conclusive diagnosis as the header of the first patient's pulse oximeter data.

Next, the pulse oximeter device's processor may execute an algorithm to simplify the first patient's pulse oximeter data, preferably by calculating statistical trend data of the raw pulse oximeter data (the first patient's acquired pulse oximeter data) (step 218). The statistical trend data may be further calculated by using moving average filtering. When the simplified pulse oximeter data and the conclusive diagnosis of the first patient's condition is displayed, the first patient may see the significant variations of the pulse oximeter data and may understand the relationship between the conclusive diagnosis and the pulse oximeter data (step 220). Different methods of simplifying the pulse oximeter data may be used, such as a smoothing filter and other types of filter.

After displaying the simplified pulse oximeter data with the conclusive diagnosis of the first patient's condition, the network patient database and the network community database are updated (step 222). Database Data—which represents or corresponds to the first patient's pulse oximeter data with the one or more markers, the conclusive diagnosis of the first patient's condition, and the simplified first patient's pulse oximeter data—are sent via the cloud network to a remote server.

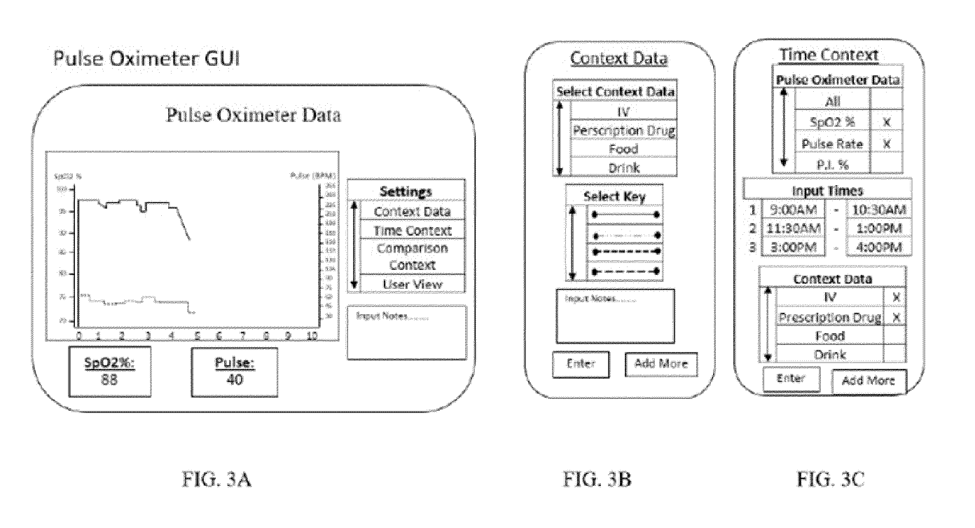

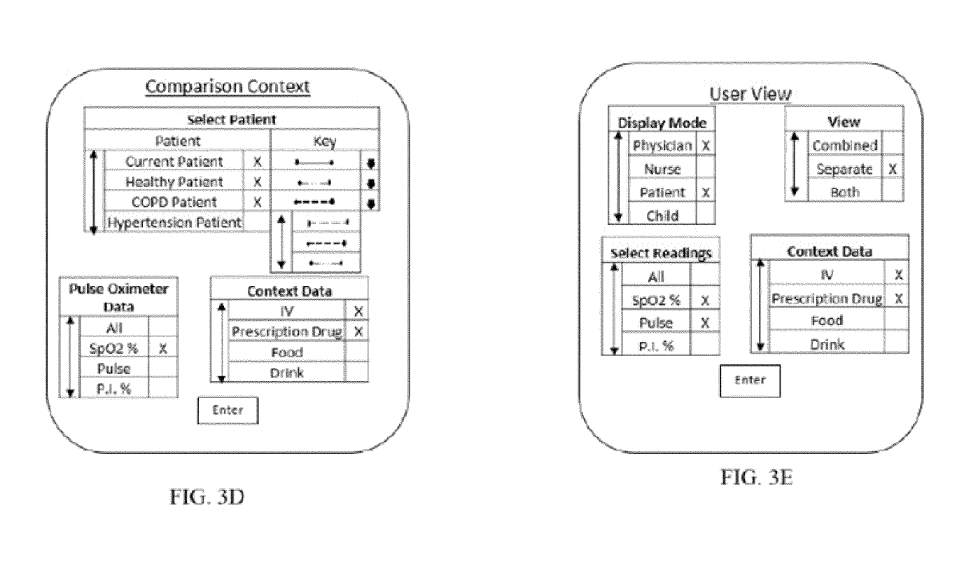

In accordance with a preferred embodiment of the present invention as illustrated in FIGS. 3A-3E, the graphical user interface of the pulse oximeter device may be used, for example, for inputting data related to pulse oximeter data and for selecting, browsing, displaying, and transferring pulse oximeter data to a remote server. As shown in FIG. 3A, the “Pulse Oximeter Data” window of the graphical user interface displays a graph of the blood oxygen saturation level (SpO2) and the pulse rate with respect to time. Under the “Settings” tab of the “Pulse Oximeter Data” window, the user can choose to execute the “Context Data” window, “Time Context” window, “Comparison Context” window, and “User View” window for configuring the display mode of the pulse oximeter data.

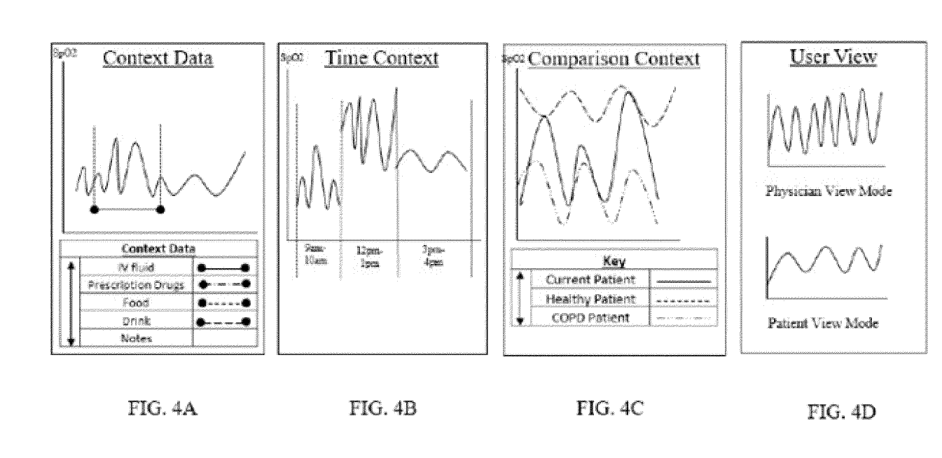

As shown in FIG. 3B, the “Context Data” window allows the user to select a marker—which may be a combination of dashes, dots, and spaces of various lengths corresponding to a selected type of context data. For example, the user selects the line segment as the marker corresponding to the administration of intravenous (IV) fluid. The graph of the pulse oximeter data is then displayed with the IV fluid marker (shown in FIG. 4A). In another embodiment of the present invention, the markers may also have variations in color and line widths.

In another window as shown in FIG. 3C, the “Time Context” window enables the user to choose the type of context data and the type of pulse oximeter data to display, which includes blood oxygen saturation percentage, pulse rate, and perfusion index percentage. The user may also choose the time period for the selected pulse oximeter data. In an exemplary embodiment of the present invention as illustrated in FIG. 4B, the user selects “9 am-11 am,” “12 pm-1 pm,” and “3 pm-4 pm” as the time periods of the pulse oximeter data to be displayed.

On the other hand, FIG. 3D shows the “Comparison Context” window that the user may use to select the type of patient—which may include current patient, healthy patient, COPD patient, and hypertension patient—wherein the pulse oximeter data of the selected type of patient is displayed. The user may further select the type of pulse oximeter data and the type of context data to be displayed. For example, the user selects “current patient,” “healthy patient,” and “COPD patient” to simultaneously display the corresponding SpO2 waveforms of each patient type (shown in FIG. 4C).

As illustrated in FIG. 3E, the “User View” window permits the user to choose the appropriate display mode. For example, the user selects the physician and patient display modes. The physician display mode shows the graph generated from the raw pulse oximeter data of the patient while the patient display mode shows the graph generated from the simplified pulse oximeter data (shown in FIG. 4D).

The communication module of the pulse oximeter device is preferably wireless such as Bluetooth, Wi-Fi, WiMax, radio frequency (RF), Zigbee, and Visual Light Communications (VLC) for enabling transmission of pulse oximeter data to the remote server.

In accordance with one aspect of the present invention, the pulse oximeter device is built with one or more internal data storage devices for storing pulse oximeter data. Alternatively, the pulse oximeter device is built with a data storage card slot that can be inserted with a removable memory device such as a secure digital (SD) card. The data storage device card slot can allow different sizes of the SD card such as the standard size, the mini size, or the micro size.

In accordance with an embodiment of the present invention, the display unit of the pulse oximeter device is preferably a flexible display, such as an LCD display, electronic paper, OLED display, electroluminescent display and other power-efficient display technology.

In accordance with the present invention, the power supply is preferably a battery integrated into the pulse oximeter device. The power supply may be a flexible type such as a lithium-polymer power supply. The power supply may be a combined rechargeable and flexible type with a self-rechargeable capability such as a flexible lithium-polymer power supply with thin-film organic solar cells. Thus, the power supply can be recharged via exposure to light. The power supply may also be removable from the pulse oximeter device.

The present invention is not intended to be restricted to the several exemplary embodiments of the invention described above. Other variations that may be envisioned by those skilled in the art are intended to fall within the disclosure.

1. A system for comparing a first patient's pulse oximeter data with a second patient's pulse oximeter data previously stored in a database, the system comprising:
a pulse oximeter device comprising:
a memory having a pulse oximeter patient database stored thereon,
a power supply;
a communications module;
one or more light emitting diodes (LEDs);
an alarm generating element;
a pulse oximeter module that acquires the first patient's pulse oximeter data;
one or more sensors;
a clock;
a display unit including a user interface for enabling a user to input one or more markers to first patient's acquired pulse oximeter data and one or more timestamps corresponding to the one or more markers; and
a processor that executes an algorithm to simplify the first patient's acquired pulse oximeter data to allow the first patient to understand the acquired pulse oximeter data;
a remote server connected to a cloud network comprising: a network base software, a network patient database, and a network community database; and
a cloud network connected to the pulse oximeter device via the communications module and the remote server, the cloud network allowing communication between the remote server and the pulse oximeter device,
wherein the pulse oximeter patient database includes database data corresponding to a diagnosed first patient's condition, the first patient's acquired pulse oximeter data having the one or more markers, and the simplified first patient's acquired pulse oximeter data.

2. The system of claim 1, wherein the pulse oximeter module is attachable to a body part of the first patient.

3. The system of claim 1, wherein the first patient's pulse oximeter data is selected from a blood oxygen saturation level, a perfusion index, a pulse rate, and a combination thereof.

4. The system of claim 1, wherein the one or more markers indicate context data of the first patient selected from a type of food administered to the first patient, a quantity of food administered to the first patient, a drug administered to the first patient, a fluid administered to the first patient, and a combination thereof.

5. The system of claim 1, wherein the display unit displays the first patient's acquired pulse oximeter data as a time-dependent graph.

6. The system of claim 1, wherein the display unit allows the user to select the second patient's pulse oximeter data.

7. The system of claim 6, wherein the display unit displays the second patient's pulse oximeter data along with the first patient's acquired pulse oximeter data having the one or more markers.

8. The system of claim 7, wherein the display unit enables the user to input a conclusive diagnosis of the first patient.

9. The system of claim 1, wherein the algorithm enables calculating a statistical trend data of the first patient's acquired pulse oximeter data.

10. The system of claim 9, wherein the statistical trend data is calculated by using a filter, the filter being at least one of a moving average filter and a smoothing filter.

11. (canceled)

12. The system of claim 8, wherein the remote server receives the database data and updates the network patient database, the network community database, or both.

13. The system of claim 1, wherein at least one of:
the communications module is a wireless communications module;
the memory is an internal data storage device;
the memory is a removable memory device;
the display unit is a flexible display
the power supply is a battery integrated into the pulse oximeter device; and
the power supply is a flexible lithium-polymer power supply with a thin-film organic solar cell.

14. (canceled)

15. (canceled)

16. (canceled)

17. (canceled)

18. (canceled)

19. A method for comparing a first patient's pulse oximeter data with a second patient's pulse oximeter data previously stored in a database, the method comprising:
acquiring a first patient's pulse oximeter data using a pulse oximeter device having a user interface;
inputting via the user interface one or more markers for one or more segments of the first patient's acquired pulse oximeter data;
displaying the first patient's acquired pulse oximeter data with the one or more markers;
storing the first patient's acquired pulse oximeter data having the one or more markers in a pulse oximeter patient database of the pulse oximeter device;
selecting via the user interface the second patient's pulse oximeter data previously stored in the database, wherein the second patient's pulse oximeter data corresponds to a known condition;
diagnosing a first patient's condition based on the first patient's acquired pulse oximeter data having the one or more markers and the second patient's pulse oximeter data;
simplifying the first patient's acquired pulse oximeter data to allow the first patient to understand the acquired pulse oximeter data;
displaying the diagnosed first patient's condition and the simplified first patient's acquired pulse oximeter data on the pulse oximeter device; and
updating the database by uploading database data corresponding to the diagnosed first patient's condition, the first patient's acquired pulse oximeter data with the one or more markers, and the simplified first patient's acquired pulse oximeter data.

20. (canceled)

21. The method of claim 19, further comprising inputting via the user interface one or more timestamps corresponding to the one or more markers.

22. The method of claim 19, wherein the one or more markers indicate context data of the first patient selected from a type of food administered to the first patient, a quantity of food administered to the first patient, a drug administered to the first patient, a fluid administered to the first patient, and a combination thereof.
